(19) United States
(12) Patent Application Publication
(10) Pub. No.: US 2006/0183072 A1
(43) Pub. Date: Aug. 17, 2006

(54) Device for application of multiple hygienic effects

(76) Inventor: Michael Black, Foster City, CA (US)

Correspondence Address: LUMEN INTELLECTUAL PROPERTY SERVICES, INC., 2345 YALE STREET, 2ND FLOOR, PALO ALTO, CA, 94306, US

(21) Appl. No.: 11/338,442

(22) Filed: Jan. 23, 2006

Related U.S. Application Data
(63) Continuation-in-part of application No. 10/424,114, filed on Apr. 25, 2003.
(63) Continuation-in-part of application No. 10/616,367, filed on Jul. 8, 2003, now Pat. No. 6,989,023.
(63) Continuation-in-part of application No. 11/179,445, filed on Jul. 12, 2005.
Application No. 11/179,445 is a continuation-in-part of application No. 10/645,674, filed on Aug. 20, 2003, now Pat. No. 6,953,341.

(51) Int. Cl. A61C 3/00; A61H 1/00; A61B 18/18

(52) U.S. Cl. 433/29; 601/15; 606/3

(57) ABSTRACT

Hygiene applicator devices for application of two or more hygienic effects to body structures are provided with the objective to effectively apply multiple hygienic effects preferably simultaneously. The applicator includes rotating element(s) rotatable relative to the handle. Two or more light sources, producing at least two unique light treatments, are connected to the rotating element(s). A transparent hollow cover is attached to the handle and covers the light sources. At least part of the cover is transparent to the unique light treatments. The cover allows the light sources to rotate within its hollow structure. Several different embodiments are described related to a comb, a brush, a toothbrush, a toothpick, a facemask, a glove or a skin-massaging wand.

### CROSS-REFERENCE TO RELATED APPLICATIONS

This application is a continuation-in-part of U.S. Patent Applications with Ser. No. 10/424,114, filed Apr. 25, 2003, Ser. No. 10/616,367 filed Jul. 8, 2003, and Ser. No. 11/179,445 filed Jul. 12, 2005 which is a continuation in part of Ser. No. 10/645,674, filed Aug. 20, 2003, which are incorporated herein by reference for all that they disclose.

### FIELD OF THE INVENTION

This invention relates to devices capable of providing hygienic treatments through light.

### BACKGROUND

Hygiene relates to the principles of cleanliness, promotion and preservation of health or the freeing from disease-causing microorganisms. Hygienic effects can be established in different ways of which one is through the effect of light on biological structures. The light treatment can be applied to superficial structures and subcutaneous structures. The effects of light on biological structures depends on the properties of the light source (e.g. active matter, beam wavelength, continuous or impulse mode of operation), characteristics of the structures, water content, pigmentation degree, vascularization, vitality, heterogeneity, specific heat conductivity or time exposure. One of the objectives in the design of hygienic devices is to effectively apply multiple hygienic effects preferably simultaneously. Such devices would then lead to a reduction in treatment time while optimizing a comprehensive application of hygienic effects. The present invention advances the art in that direction.

### SUMMARY OF THE INVENTION

The present invention provides hygiene applicator devices for application of two or more hygienic effects to body structures with the objective to effectively apply multiple hygienic effects preferably simultaneously. The applicator includes a handle and at least one rotating element rotatable relative to the handle. Two or more light sources, producing at least two unique light treatments, are connected to the rotating element(s). A transparent hollow cover is attached to the handle and covers the light sources. At least part of the cover is transparent to the unique light treatments. The cover allows the light sources to rotate within its hollow structure. In one embodiment, the applicator could include at least one support element for the light sources. The support elements could then be connected to the rotating element(s). The hygiene treatment applicator could also include a hygiene pattern generation means for creating a pattern of hygienic light treatments. Several different embodiments are described related to a comb, a brush, a toothbrush, a toothpick, a facemask, a glove, skin-massaging wand.

### BRIEF DESCRIPTION OF THE FIGURES

The objectives and advantages of the present invention will be understood by reading the following detailed description in conjunction with the drawings, in which:

FIG. 1 shows examples of the application of multiple hygienic effects according to the present invention; and

FIGS. 2-14 show different examples of hygiene applicator devices according to the present invention.

### DETAILED DESCRIPTION OF THE INVENTION

Although the following detailed description contains many specifics for the purposes of illustration, anyone of ordinary skill in the art will readily appreciate that many variations and alterations to the following exemplary details are within the scope of the invention. Accordingly, the following preferred embodiment of the invention is set forth without any loss of generality to, and without imposing limitations upon, the claimed invention.

The present invention provides a hygienic treatment application device for applying two or more unique hygiene light treatments to body structures. These light treatments are established by two or more light sources each capable of delivering a light beam with a unique hygiene light treatment to the body structures. The light sources are preferably low power light sources including low power lasers, light emitting diodes or low power semiconductor lasers ranging from the ultraviolet, visible or infrared spectrum. The desired light treatment(s) that one would like to obtain guides the choice of the light source (light sources) and the parameter(s). By varying parameters such as e.g. fluence, spot size, mode such as continuous or pulsed, repetition rate, pulse duration different light treatments could be established.

In general, light treatments are defined as treatments with hygienic effects that relate to the cleanliness of these structures, promotion and preservation of health of the structures, freeing the body structure from disease-causing microorganisms or providing therapeutic or treatment effects. In particular, the present invention encompasses hygienic effects related to the hygienic effect of visible, near ultraviolet and infrared light on these structures, which are known in the art (for a light spectrum refer to page 13 in a book by Tuner et al. (1996) entitled “Laser therapy in dentistry and medicine” and published by Prisma Books, Grangesberg, Sweden). Examples of such hygienic effects that could be selected include anti-inflammatory effects, preventative effects, caries-protective effects, heating effects anti-bacterial effects, sterilizing effects, cleaning effects, cosmetic effects, therapeutic effects, healing effects, bio-stimulative effects, bio-altering effects, pain-releaving effects, teeth whitening effects, photo-rejuvination effects, photodynamic effects or agent-penetration effects.

To establish a particular hygienic effect at a body structure one needs to consider the light source properties such as the type of low power light source, wavelength of the light beam, the continuous or impulse mode of operation of the light sources, characteristics of the structures, water content of the structures, pigmentation degree of the structures, vascularization of the structures, vitality of the structures, heterogeneity of the structures, specific heat conductivity of the structures, the fluence of light penetration through a structure or the time exposure needed for the light beam. The art provides teachings on hygienic photo-effects of structures including guidelines regarding parameters such as the type of light source, selection of wavelength(s), fluence, penetration, selection of spot size, recommended pulse duration, recommended repetition rate, or the like. The selection of the hygienic effect as part of the present invention incorporates these teachings as well as new teachings that become available in the art describing newly identified hygienic effects.

Currently available teachings are described in the following books, which provide an exemplary list rather than a comprehensive list. The list includes a book by Goldman (1981) entitled “The biomedical laser: technology and clinical applications” and published by Springer-Verlag, New York; a book by Katzir (1993) entitled “Lasers and optical fibers in medicine” and published by Academic Press, New York; a book by Hajder et al. (1994) entitled “Acupuncture and lasers” and published by Ming, Belgrade; a book by Tuner et al. (1996) entitled “Laser therapy in dentistry and medicine” and published by Prisma Books, Grangesberg, Sweden; a book by Alster et al. (1996) entitled “Cosmetic laser surgery” and published by Wiley & Sons, New York; or a book by Fitzpatrick et al. (2000) entitled “Cosmetic Laser Surgery” and published by Mosby, St. Louis).

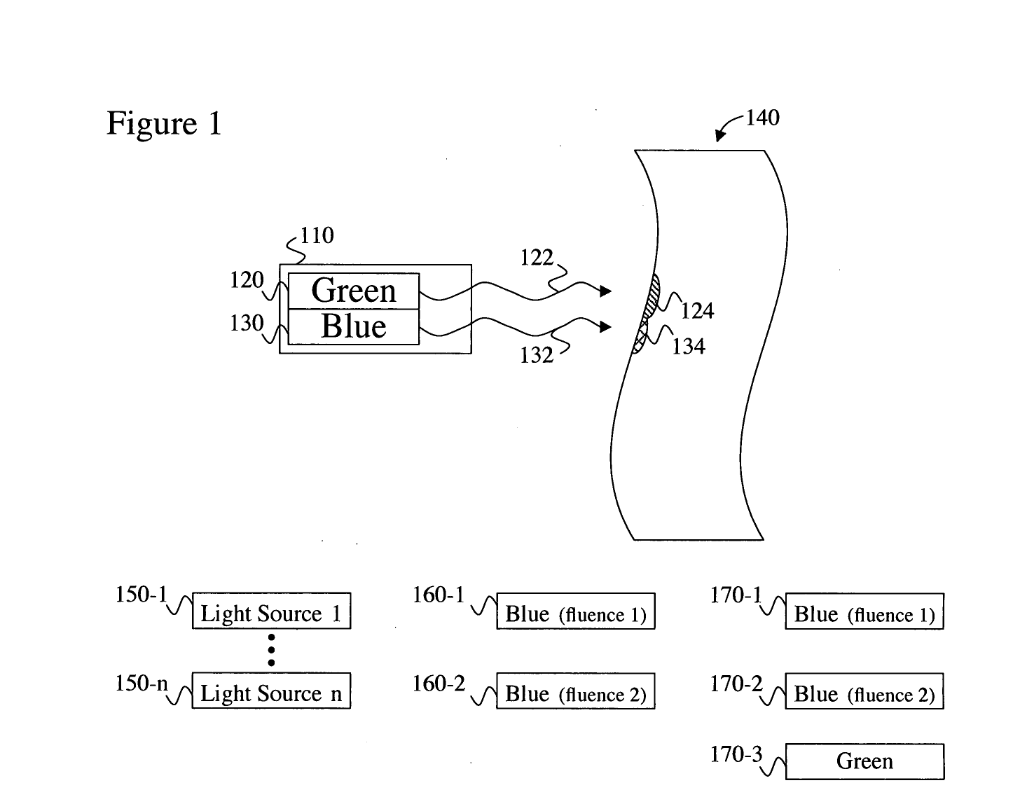

FIG. 1 shows an exemplary embodiment of an element 110 with two light sources 120, 130 dynamically delivering a light beam with a green wavelength 122 and a light beam with a blue wavelength 132, respectively. The green wavelength 122 and the blue wavelength 132 each provide a unique hygienic effect when applied to body structure 140. In this example, light beams 122, 132 have both a fairly superficial hygienic effect, yet unique and different from each other, at body structure 140 as shown by 124, 134 respectively. In general, two or more light sources could be used such as n light sources 150-1 to 150-n. Two of the same light sources could be used such as two light sources 160-1, 160-2 that each deliver blue light, however, with at least one different parameter to establish a different and unique hygienic effect for each of the two light sources 160-1, 160-2. Such a different and unique hygienic effect could be established by different fluences for each of the two light sources 160-1, 160-2, i.e. fluence 1 and fluence 2, respectively. The relative subsurface fluence of a light beam in a structure is dependent on the spot size, which could be relatively small or relatively large. The same subsurface fluence values appear at deeper levels with the larger spot size compared to the smaller spot size. Another example is that there are three light sources, of which two are the same 170-1, 170-2 and one 170-3 is different, though all three delivering a unique hygienic effect.

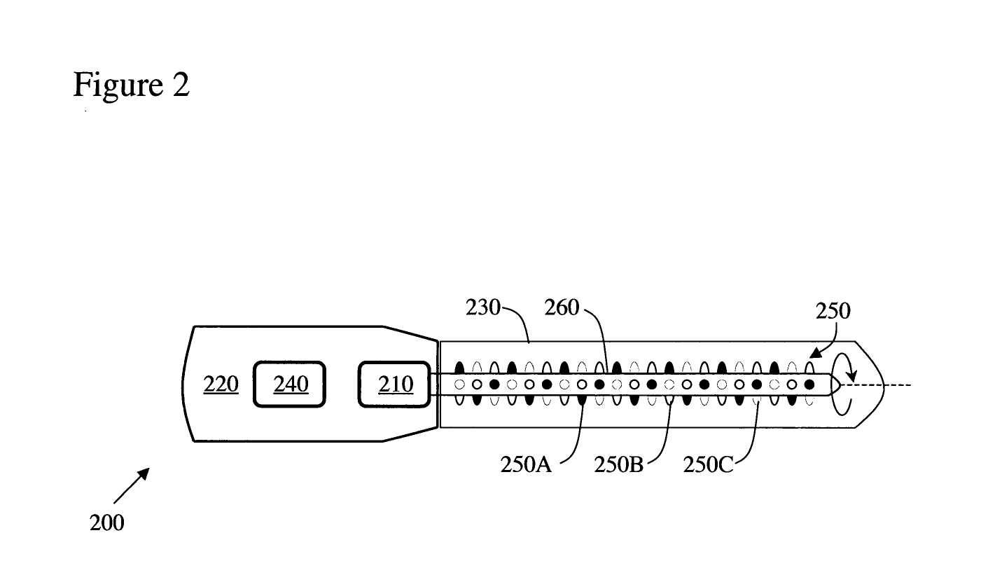

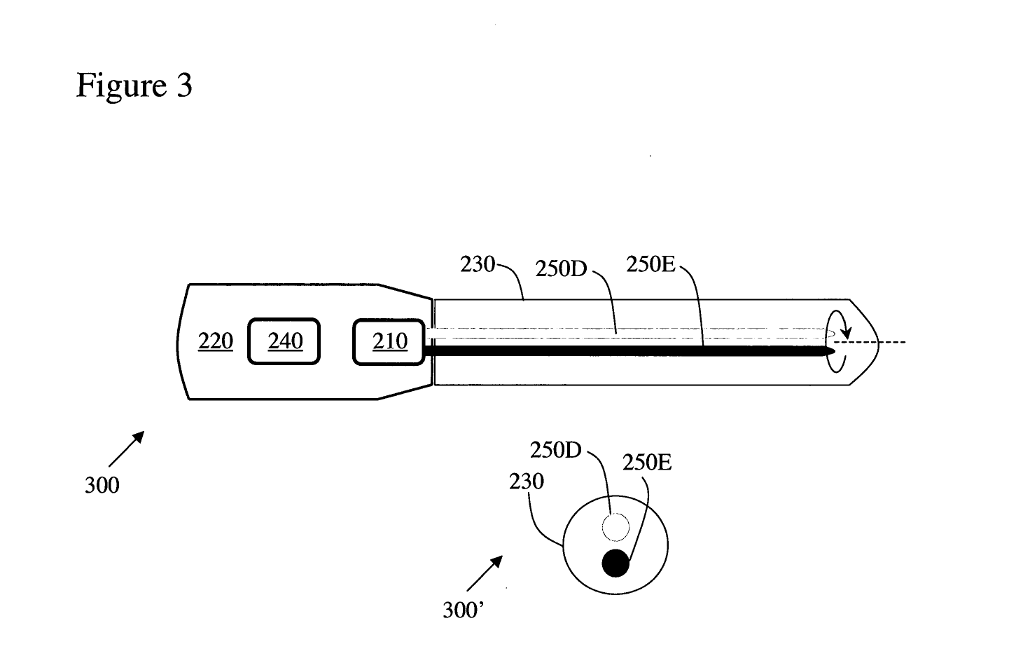

Exemplary hygiene treatment applicator devices according to the present invention are shown in FIGS. 2-14 (100, 200, 300, 400, 400′, 500, 500′, 600, 600′, 700, 700′, 800, 800′, 900, 900′, 1000, 1110, 1120 and 1130). It is noted that these are examples and that the invention is not limited to these devices. One of the key ideas of each of hygiene treatment applicator is that they have at least one rotating element 210 rotatable relative to a handle 220. Examples of rotating elements are electric motors, spinning wheels, rotating wheel or the like. Two or more light sources 250 (250A-E) are connected to rotating element(s) 210. As indicated supra the light sources produce at least two unique light treatments. In one example in FIG. 2, three light sources 250A-C are shown each with unique light treatments. In another example shown in FIG. 3, two light sources 250D-E are shown that could either be short, long or extended light sources each with unique light treatments (see FIG. 2).

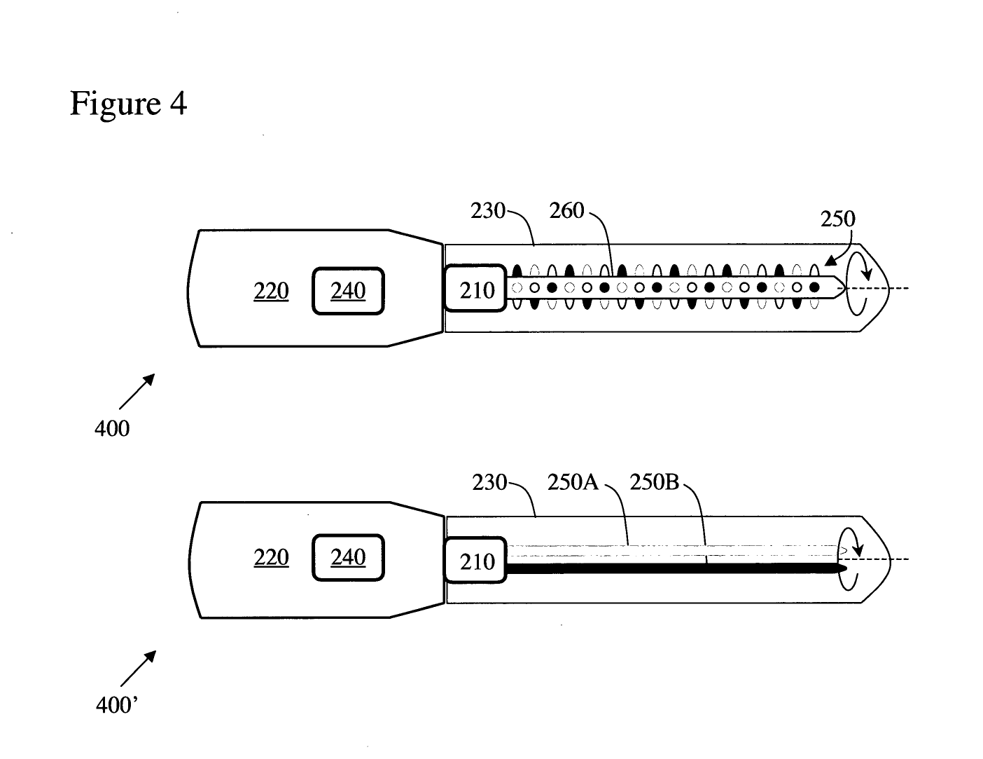

The light sources could be directly connected to the rotating element as shown in FIGS. 3 (300) and 4 (400′). In another embodiment, a support element 260 for the light sources is included that is connected to the rotating element(s) as shown in FIGS. 2, 4 (400), 7 (700′) and 8 (800′).

A transparent hollow cover 230 is included and placed over at least the light sources 250. Cover 230 is connected to handle 220. It is important though that at least part of cover 230 is transparent to the unique light treatments and that the light sources are able to rotate within the cover. Cover could be a soft plastic, a hard plastic, a silicone, a latex, a PTFE (polytetrafluoroethylene), pyrex, glass, or a polyurethane.

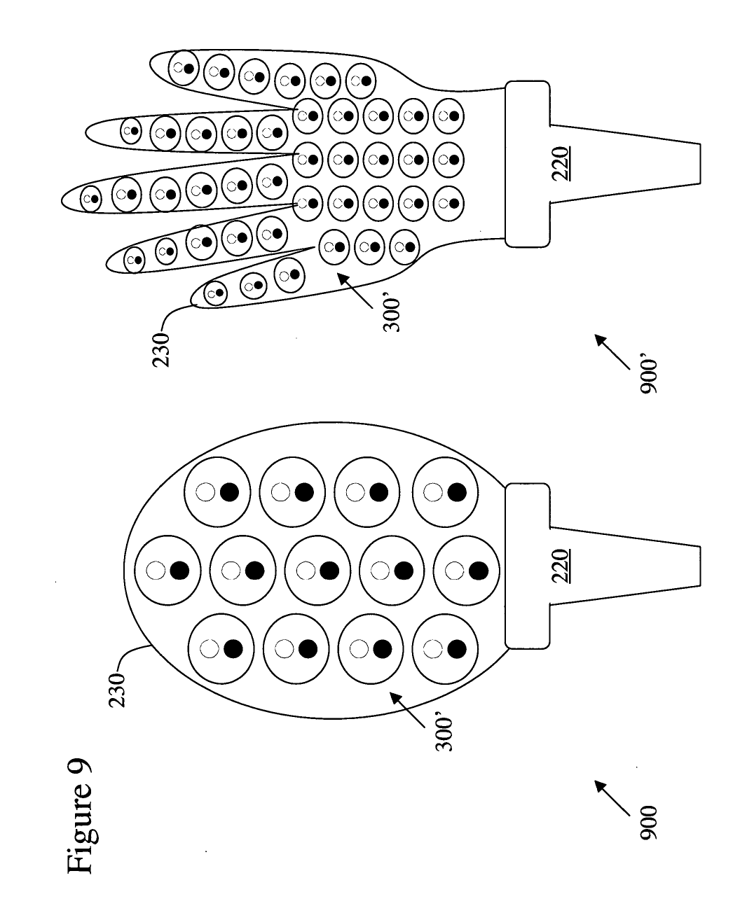

Rotating element(s) 210 could be housed inside handle 220 as shown in e.g. FIGS. 2-3 or could be housed inside cover 230 as shown in e.g. FIGS. 4 and 9.

The idea of having light sources 250 connected to rotation element 210 is that when in use, the lights sources are rotating around an axis (e.g. the longitudinal axis as shown in FIGS. 2-4 and 7-8) relative to handle 220. Due to rotation, body structures in proximity or in contact with cover 230 (i.e. light sources) receive a plurality of unique light beams. Enhancing the multiple treatment effect is achieved by distributing light sources with different hygiene effects inside the cover 230 or over support element 260. The user moving the device relative to the body structure(s) further enhances the multiple treatment effect.

The hygiene treatment could also be achieved by having a hygiene pattern generation means 240 for creating a pattern of hygienic light treatments. The hygiene pattern generation means generates predetermined hygienic patterns, random hygienic patterns or user-selected hygienic patterns. Varying the parameters as discussed supra could create patterns.

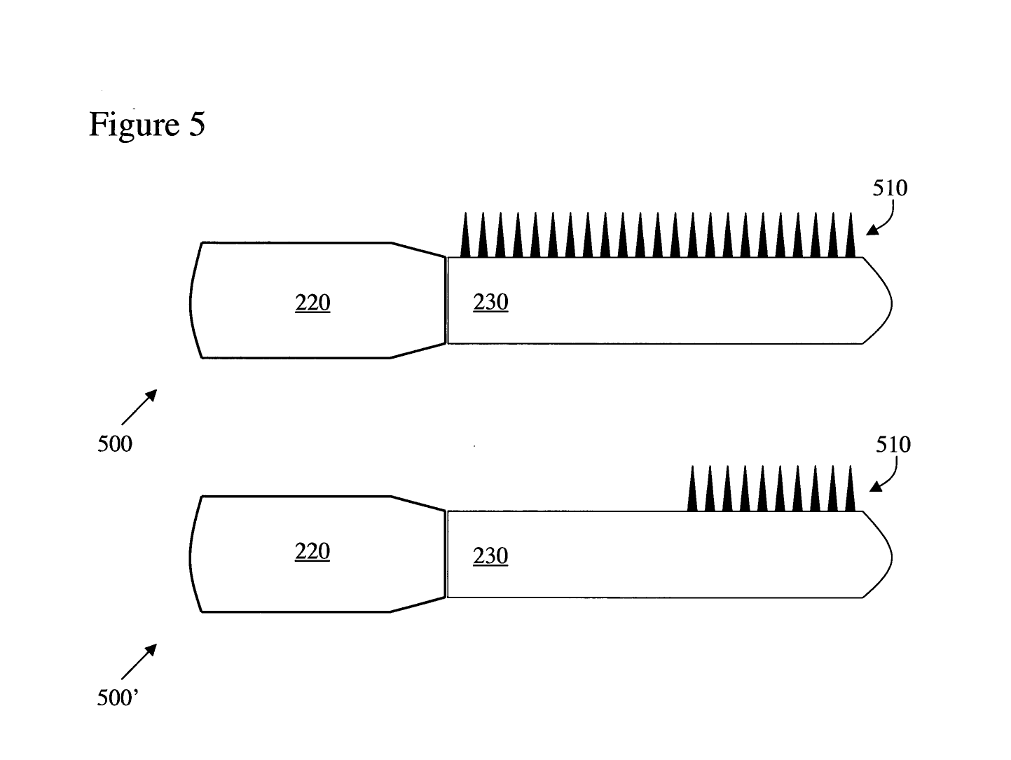

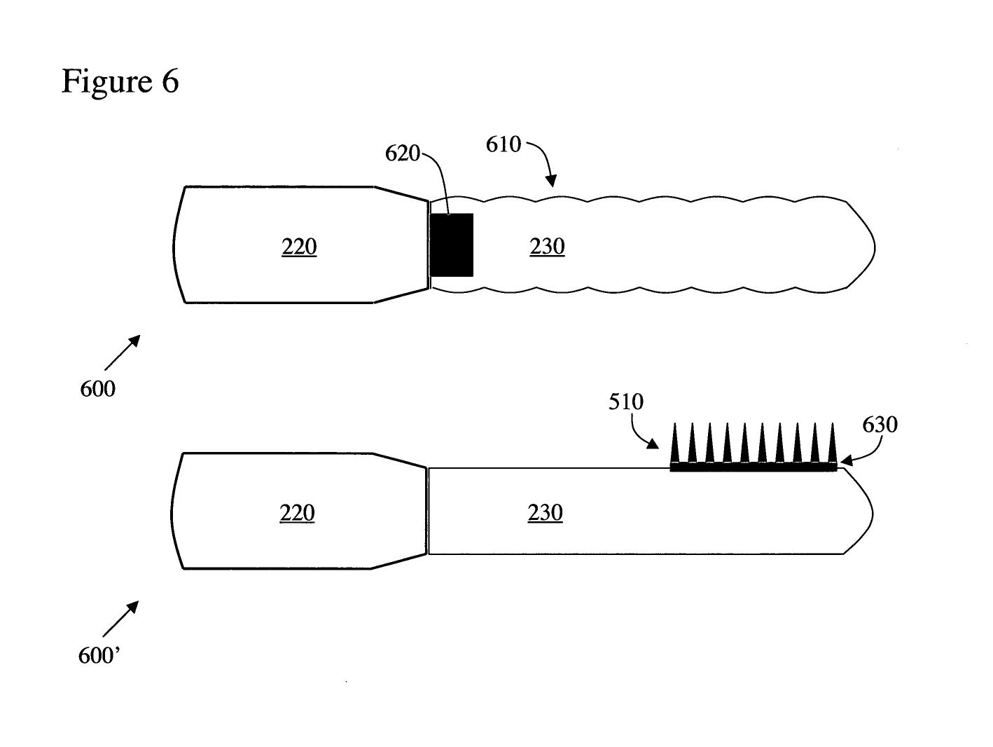

Dependent on the type of use of the hygiene applicator, cover 230 could take different shapes. For example cover 230 could be elongated, long, round, tapered, bead-shaped, flat, spherical, circular, dome-like, concave, convex, or any combination thereof. Cover 230 could also have a plurality of bristles, a plurality of teeth, a plurality of flexible filaments, a plurality of stiff filaments, or the like (see 510 in FIG. 5). The surface of cover 230 could also have texture 610 as shown in FIG. 6 (600). Texture could be a rippled surface, bumped surface, surface with bulges, surface with domes, etc. In one embodiment, all of these additions to cover 230 (bristles, teeth, filaments, textures, or the like) could be transparent to the light treatments just like cover 230.

If desired, one could include one or more light focusing elements, one or more light diffracting elements, one or more reflective coatings or one or more claddings e.g. to the cover (surface or inside) to respectively focus light, diffract light or prevent light beams from going through particular parts of the cover. Examples of such focusing elements, light diffracting elements, reflective coatings or claddings are known in the art and the selection depends on the type of light beams as a person of average skill in the art would readily appreciate.

In one example, cover 230 could include texture with optical capabilities such as focusing or diffracting the light beams. For example, the texture could be concave, concave, gratings or any optical element that could affect the light beam. Furthermore, each cover could include various combinations of optical elements to affect the light beam and is not restricted to one particular type.

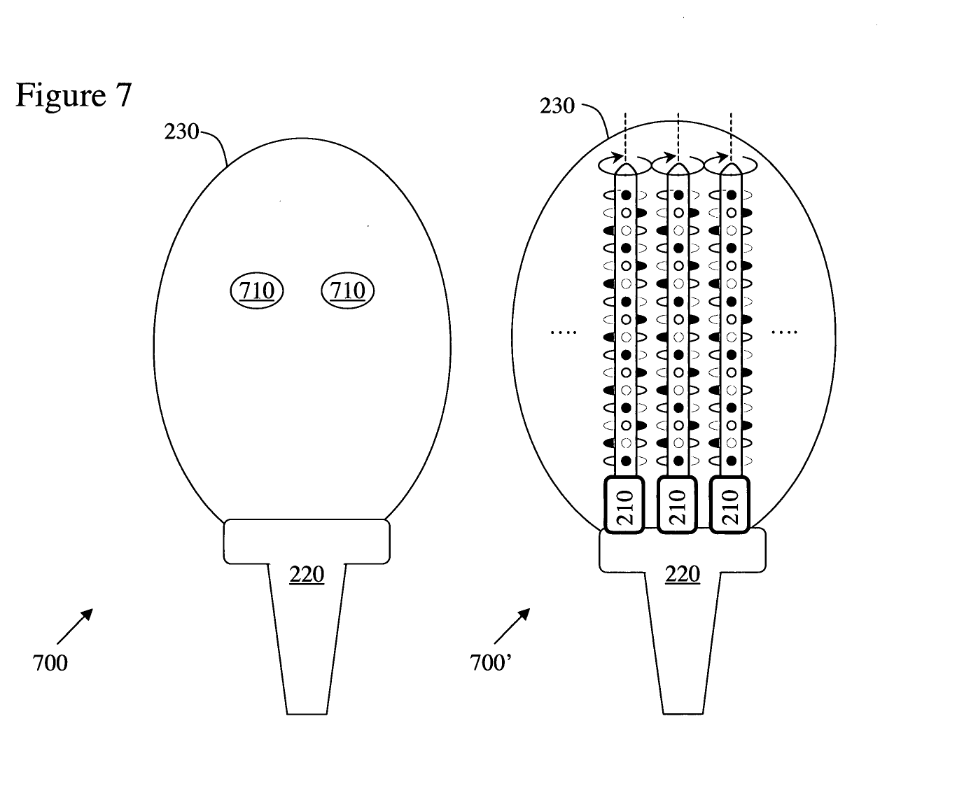

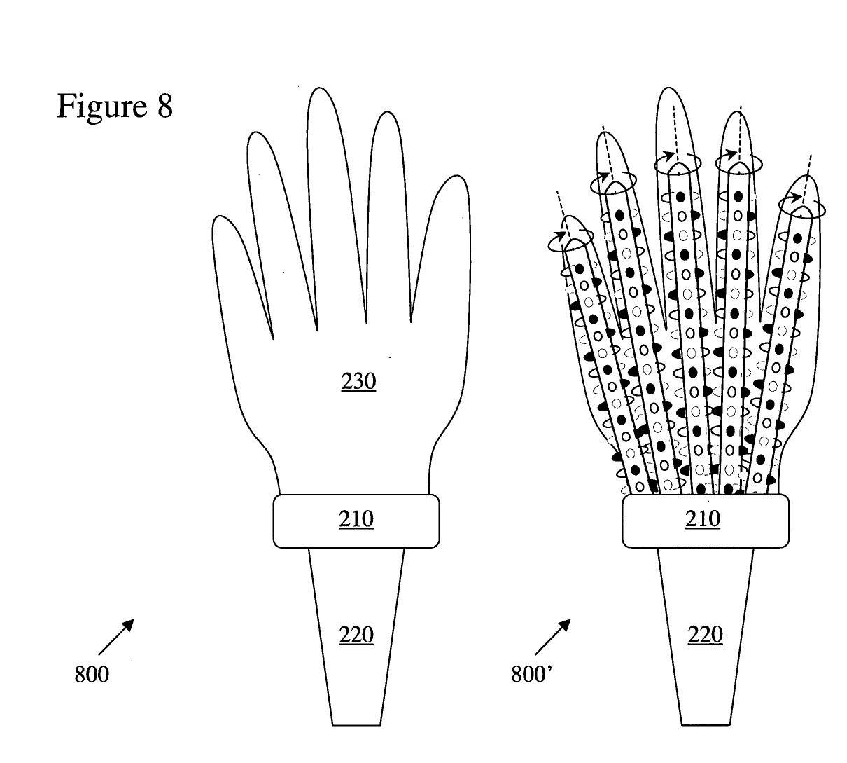

By changing the size and shape of cover 230, different hygiene applicator can be achieved, for example the reader is referred to U.S. Patent Applications with Ser. No. 10/424,114, filed Apr. 25, 2003, Ser. No. 10/616,367 filed Jul. 8, 2003, Ser. No. 10/645,674, filed Aug. 20, 2003 and Ser. No. 11/179,445 filed Jul. 12, 2005 all by the same inventor as the present invention, which are all incorporated by reference for all that they disclose. For example, at least part of cover 230 could be the head of a comb, a brush, a toothbrush, a toothpick, a spider with multiple arms/tentacles, a skin-massaging wand, or the like (see e.g. FIGS. 5, 500 and 500′). In another embodiment, at least part of cover 230 is a facemask possible with opening 710 for eyes (see e.g. FIGS. 7, 700 and 700′). In yet another embodiment, at least part of cover 230 is a glove (see e.g. FIGS. 8, 800 and 800′). Cover 230 could be a hollow cover on one side of a hand or on both sides of a hand. In yet another embodiment shown in FIG. 9, cover 230 could include multiple rotating elements 300′ each with two or more light sources which in this example are capable of rotating along the axis (not shown) perpendicular to the drawing.

FIG. 6 shows two embodiments 600, 600′ in which a vibrating means 620, 630 or a massaging means is included in the device with the objective to provide vibration to cover 230 or, additionally, in some cases, to the base of the teeth, bristles or filaments 510. Examples of vibrating or massaging means that could be used are an ultrasonic means, a piezoelectric means or a mechanical means, all which are known in the art. Vibrating means could be housed inside the handle, inside the cover, within the cover or at the surface of the cover.

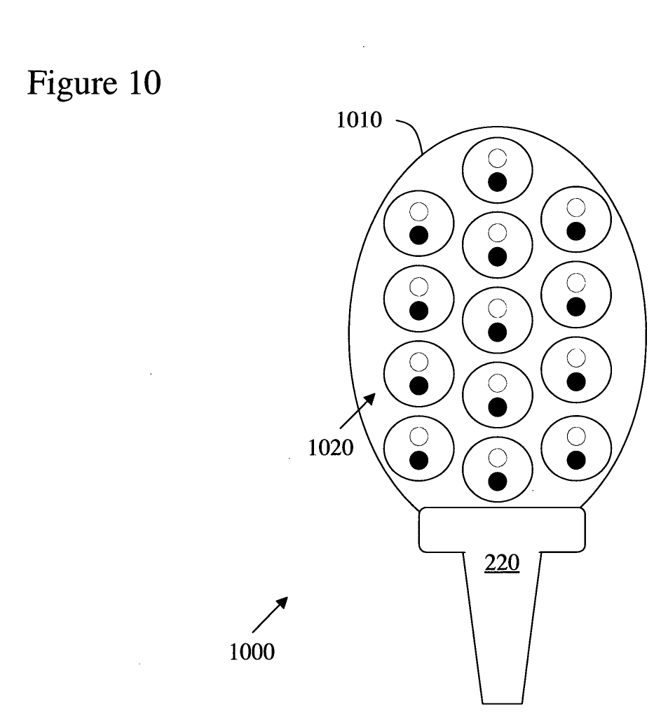

The present invention has now been described in accordance with several exemplary embodiments, which are intended to be illustrative in all aspects, rather than restrictive. Thus, the present invention is capable of many variations in detailed implementation, which may be derived from the description contained herein by a person of ordinary skill in the art. For example, handle, light sources, cover and/or support elements could either fixed or removably connected to each other. Another variation is that the device could have multiple covers and is not limited to one cover. For example, embodiment 1000 shown in FIG. 10 could have a support surface 1010 for multiple sets 1020′ whereby each set 1020′ is now containing a rotating element, at least one light source and a cover. Sets 1020′ could have different types of light sources with unique treatment effects and distributed over the surface.

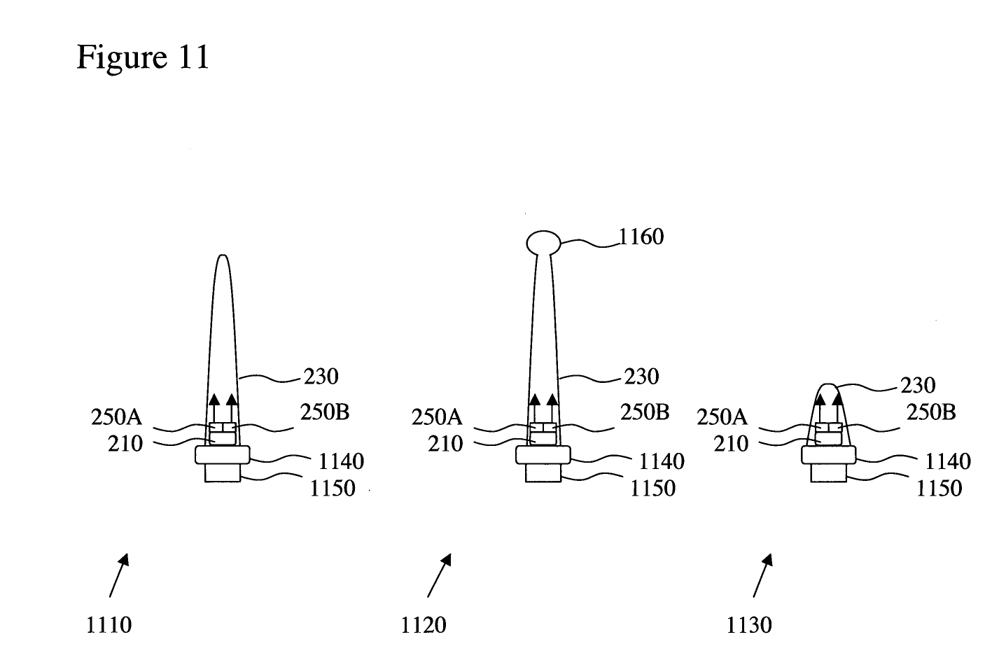

FIG. 11 shows another variation with examples of elements 1110, 1120, 1130 similar to the teaching of U.S. patent application Ser. No. 11/179,445 filed Jul. 12, 2005 with the difference that the examples here in FIG. 11 include a rotating element 1140 at the base of the light sources, e.g. 250A, 250B. Each element 1110, 1120 and 1130 has a cover 230, which defines the shape or size of the element. Light sources, 250A, 250B are situated near the bottom and inside cover 230. The elements further distinguish a base 1140 that supports cover 230, and is further integrated with a connector part 1150. Connector part 1150 fits the support like a male/female connector. The art teaches many different mechanisms for connector part 1150 all which are useful to this invention. Cover 230 could further be a bead-shape cover as indicated by the bead-shape 1160 in example 1120.

Similar to the teachings in of U.S. patent application Ser. No. 11/179,445 filed Jul. 12, 2005, dependent on the type of treatments and/or preferred types of elements a user could create a pattern of elements with: (i) elements providing different hygienic effects, and/or (ii) elements having different shapes or sizes. By having removable or detachable elements, the user is capable of changing the pattern and creating a new topographical surface as desired for his/her hygienic treatment plan. The (flexible) support could take the shape of a glove, a facemask, or other suitable devices used for hygiene or treatment application. In case of a glove the elements are (preferably) removably attached to the outside of the glove so that a user could e.g. rub his/her face and apply the hygienic treatments. In case of a facemask the elements are (preferably) removably attached to the inside of the facemask so that a user wear the facemask and apply the hygienic treatments. Examples of flexible supports are, for instance, but not limited to, latex, silicone, rubber, PTFE (polytetrafluoroethylene), polyurethane, or the like.

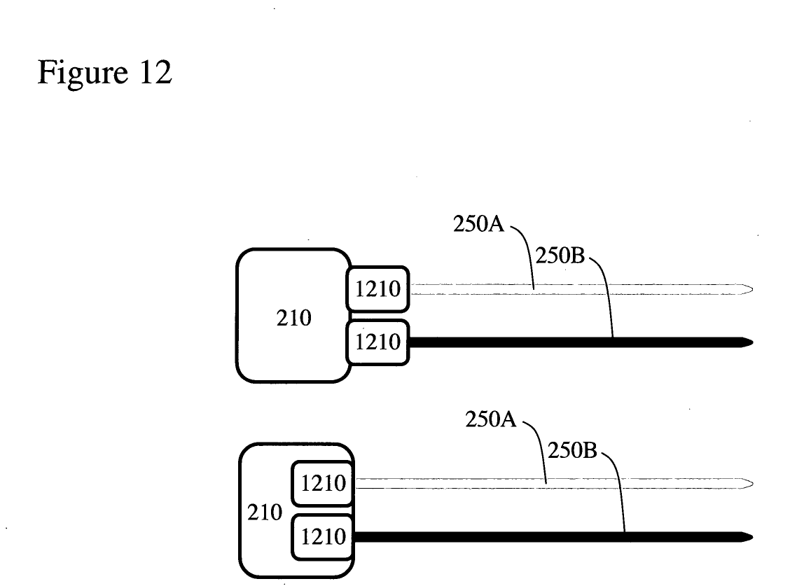

Another variation relates to rotating element 210. So far we discussed that each rotating element rotates all light sources connected to the rotating element. However, each light source could also include its own element 1210 that could either cause motion, rotation and/or vibration (e.g. a solenoid, electric motor, piezoelectric device or the like) as shown in two examples in FIG. 12. Element 1210 could be placed at the base of the light source 250A in between the light source and the rotating element 210, or could be an integral part of rotating element. In this case, each light source connected to rotating element 210 could now also move/vibrate/rotate independently from the other light sources connected to rotating element 210. It is noted that these movements are not restricted to rotation but could be any translational or rotational movement with respect to the rotating element or base. Furthermore the movements could also be any combination of translations and rotations. In general the movements as discussed herein could be in any direction, degree of rotation or any combination as desired by the user or manufacturer.

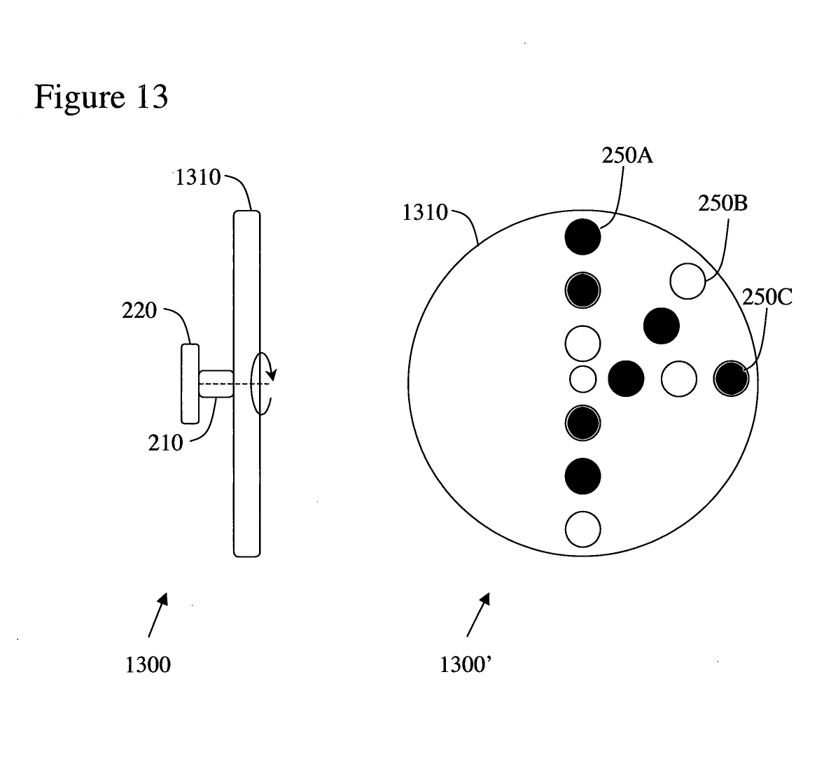

Still another variation is shown in FIG. 13 where a support surface 1310 has attached multiple light sources, e.g. 250A-C (1300 is a side view and 1300′ is a top view of the same). The light sources could be distributed at various angles or radii (some of which illustrated in 1300′). The key idea here is that multiple light sources with different unique treatment effects circle around the axis as shown. This example is not limited to the type of distribution, number of light sources, or type of light sources.

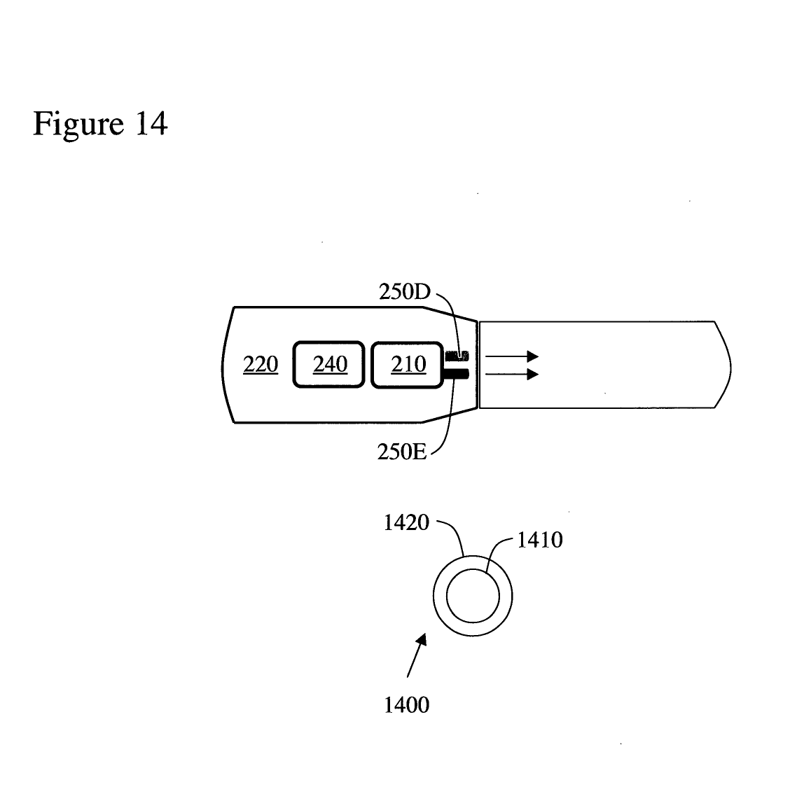

Still another variation pertains to where the rotating element(s) and light sources are located. In the variation shown in FIG. 14, the rotating element(s) 210 and light sources 250 are located inside the handle 220. Handle 220 now would have a transparent window 1410 to allow transmission of the light beams (indicated by arrows) into sleeve or cover 230. Note that the bottom part of FIG. 14 is a frontal view of handle 220 showing the face 1420 with transparent window 1410 that meets sleeve or cover 230.

All such variations are considered to be within the scope and spirit of the present invention as defined by the following claims and their legal equivalents.

What is claimed is:

1. A hygiene treatment applicator for application of two or more hygienic effects, comprising:
(a) a handle;
(b) a rotating element rotatable relative to said handle;
(c) two or more light sources fixed or remobably connected to said rotating element, wherein said light sources produces at least two unique light treatments; and
(d) a transparent hollow cover over said light sources, wherein said cover is fixed or remobably connected to said handle, wherein at least part of said cover is transparent to said unique light treatments and wherein said light sources are rotating within said cover.

2. The hygiene treatment applicator as set forth in claim 1, further comprising at least one support element for said light sources, wherein each one of said support elements is fixed or removably connected to said rotating elements.

3. The hygiene treatment applicator as set forth in claim 1, wherein said cover is elongated, long, round, tapered, bead-shaped, flat, spherical, circular, dome-like, concave, convex, or any combination thereof.

4. The hygiene treatment applicator as set forth in claim 1, wherein at least part of the surface of said cover comprises texture.

5. The hygiene treatment applicator as set forth in claim 1, wherein at least part of the surface of said cover comprises a plurality of bristles, a plurality of teeth, a plurality of flexible filaments or a plurality of stiff filaments.

6. The hygiene treatment applicator as set forth in claim 1, wherein said cover is a soft plastic, a hard plastic, a silicone, a latex, a PTFE (polytetrafluoroethylene), pyrex, glass or a polyurethane.

7. The hygiene treatment applicator as set forth in claim 1, wherein said two or more light sources are low power lasers, light emitting diodes or semiconductor lasers.

8. The hygiene treatment applicator as set forth in claim 1, wherein the light beams comprise light from the ultraviolet, visible or infrared spectrum.

9. The hygiene treatment applicator as set forth in claim 1, wherein said handle further comprises a vibrating means to vibrate said cover.

10. The hygiene treatment applicator as set forth in claim 1, wherein said cover further comprises a vibrating means to vibrate said cover.

11. The hygiene treatment applicator as set forth in claim 5, wherein said cover further comprises a vibrating means to vibrate said plurality of bristles, said plurality of teeth, said plurality of flexible filaments or said plurality of stiff filaments.

12. The hygiene treatment applicator as set forth in claim 1, wherein at least part of said cover is the head of a comb.

13. The hygiene treatment applicator as set forth in claim 1, wherein at least part of said cover is the head of a brush.

14. The hygiene treatment applicator as set forth in claim 1, wherein at least part of said cover is the head of a toothbrush.

15. The hygiene treatment applicator as set forth in claim 1, wherein at least part of said cover is the head of a toothpick.

16. The hygiene treatment applicator as set forth in claim 1, wherein at least part of said cover is a facemask.

17. The hygiene treatment applicator as set forth in claim 1, wherein at least part of said cover is a glove.

18. The hygiene treatment applicator as set forth in claim 1, wherein at least part of said cover is a skin-massaging wand.

19. The hygiene treatment applicator as set forth in claim 1, further comprising a hygiene pattern generation means for creating a pattern of hygienic light treatments, wherein said hygiene pattern generation means generates predetermined hygienic patterns, random hygienic patterns or user-selected hygienic patterns.

20. The hygiene treatment applicator as set forth in claim 1, further comprising one or more focusing elements, one or more light diffracting elements, one or more reflective coatings or one or more claddings.

21. A hygiene treatment applicator for application of two or more hygienic effects, comprising:
(a) a handle;
(b) at least one rotating element each rotatable relative to said handle;
(c) a plurality of light sources fixed or remobably connected to each of said rotating element, wherein said light sources produces at least two unique light treatments; and
(d) a transparent hollow cover over said light sources, wherein said cover is fixed or remobably connected to said handle, wherein at least part of said cover is transparent to said unique light treatments and wherein said light sources are rotating within said cover.

22. The hygiene treatment applicator as set forth in claim 21, further comprising at least one support element for said light sources, wherein each of one of said support elements is fixed or removably connected to one of said rotating elements.
